(19) United States
(12) Patent Application Publication
(10) Pub. No.: US 2026/0083767 A1
(43) Pub. Date: Mar. 26, 2026

(54) SOLID DOSAGE FORM OF A SMALL MOLECULE ANTIVIRAL AND USES THEREOF

(71) Applicant: Merck Sharp & Dohme LLC, Rahway, NJ (US)

(72) Inventors: Nicole Devitt, South Plainfield, NJ (US); Brandon G. Gerberich, Rahway, NJ (US); Prapti Kafle, Rahway, NJ (US); Michael Lowinger, Ridgewood, NJ (US); Grace A. Okoh, Chalfont, PA (US); Sanjaykumar Patel, Roselle Park, NJ (US); Somya Singh, Jersey City, NJ (US); James S. Tarabokija, Tenafly, NJ (US); Yuejie Zhao, Edison, NJ (US)

(73) Assignee: Merck Sharp & Dohme LLC, Rahway, NJ (US)

(21) Appl. No.: 19/137,855

(22) PCT Filed: Dec. 11, 2023
(86) PCT No.: PCT/US2023/083271

§ 371 Date: Jun. 11, 2025

Related U.S. Application Data
(60) Provisional application No. 63/433,155, filed on Dec. 16, 2022.

(51) Int. Cl. A61K 31/7068; A61K 9/16; A61K 9/48

(52) U.S. Cl. CPC A61K 31/7068; A61K 9/16; A61K 9/1611; A61K 9/1617; A61K 9/1652; A61K 9/4808; A61K 9/4816; A61K 9/4858; A61K 9/4866

(57) ABSTRACT

The present disclosure relates to oral dosage forms of pharmaceutical formulations that comprise an antiviral nucleoside, one or more compression aids, one or more glidants, and one or more lubricants, and one or more disintegrants, wherein the oral dosage form is a pellet having a diameter of less than or equal to 4.0 mm.

### CROSS-REFERENCE TO RELATED APPLICATIONS

This international application claims the benefit of priority to U.S. Provisional Application No. 63/433,155, filed Dec. 16, 2022, the entirety of which is incorporated herein by reference.

### FIELD

The present disclosure relates to solid dosage formulations of a small molecule antiviral. The solid dosage formulations of the disclosure are typically tablets, including pellets, mini-tablets, and granules. The present disclosure also relates to uses and methods of using the solid dosage forms for the treatment or prevention of viral infection in a patient in need thereof.

### BACKGROUND

Viral infections, such as infections caused by Eastern Equine Encephalitis Virus (EEEV), Western Equine Encephalitis Virus (WEEV), and Venezuelan Equine Encephalitis Virus (VEEV), Chikungunya fever virus (CHIK), Ebola virus, influenza virus, respiratory syncytial virus (RSV), Zika virus, and coronaviruses, such as Severe Acute Respiratory Syndrome Coronavirus (SARS-CoV), Middle East Respiratory Syndrome Coronavirus (MERS-CoV), and, most recently, SARS-CoV-2 (also known as 2019-nCoV), continue to cause illnesses, which can be mild to severe to life-threatening and fatal, across the globe.

EEEV, WEEV, VEEV, and CHIK virus are vector-borne viruses (family Togaviridae, genus Alphavirus) that can be transmitted to humans through mosquito bites. The equine encephalitis viruses are CDC Category B pathogens, and the CHIK virus is Category C.

Coronaviruses cause a large percentage of respiratory illnesses in humans, which can be severe or life-threatening. SARS-CoV-1, which emerged in 2002, has caused at least 8439 human illnesses globally and at least 812 deaths (WHO Cumulative Number of Reported Probable Cases of SARS, From 1 Nov. 2002 To 4 Jul. 2003, downloaded from www.who.int/csr/sars/country/2003_07_04/en/, on Aug. 12, 2020). Similarly, MERS-CoV emerged in 2012 and has caused at least 2519 human illnesses globally and at least 866 deaths (WHO Middle East respiratory syndrome, MERS situation update, January 2020, downloaded from www.emro.who.int/health-topics/mers-cov/mers-outbreaks.html, on Aug. 12, 2020). More recently, SARS-CoV-2 emerged in 2019, and it has caused at least 629 million confirmed human illnesses globally and at least 6.5 million deaths (World Health Organization COVID-19 Weekly Epidemiological Update, Issue No. 117, published 9 Nov. 2022, downloaded from www.who.int/publications/m/item/weekly-epidemiological-update-on-covid-19---9-november-2022, on Nov. 10, 2022). SARS-CoV-2 causes the disease referred to as COVID-19, which can include severe respiratory disease and systemic disease manifestations in humans. SARS-CoV-2 infection is also associated with mental and neurological symptoms that can include delirium or encephalopathy, agitation, stroke, meningo-encephalitis, impaired sense of smell or taste, anxiety, depression, and sleep problems, and these neurological symptoms can occur in the absence of respiratory symptoms (see Clinical management of COVID-19 (Interim guidance, 27 May 2020), downloaded from www.who.int/publications/i/item/clinical-management-of-covid-19, on Sep. 15, 2020). Additional studies are needed to further characterize the SARS-CoV-2 virus and to identify ways to prevent and treat the COVID-19 disease, as well as diseases caused by other human coronaviruses.

Many patients with COVID-19 recover with no or minimal medical intervention. However, clinical progression to severe disease severely impacts both patients and healthcare systems, increasing an individual's risks of requiring mechanical ventilation and of death and potentially overburdening hospital capacity and available healthcare resources during COVID-19 surges. Reducing the number of patients requiring hospitalization for COVID-19 is therefore critical. Vaccination remains by far the most important medical intervention available to reduce the risk of hospitalization or death from COVID-19. However, early treatment soon after symptom onset has also been shown as effective. The monoclonal antibodies bamlanivimab/etesevimab, casirivimab/imdevimab, and sotrovimab are currently the only treatments authorized for at-risk outpatients with COVID-19. Because monoclonal antibodies require administration via infusion or injection in a medical setting, a direct-acting, oral agent that can be administered at home after diagnosis may be more practical for non-hospitalized patients and would be an important new tool in treating COVID-19 caused by SARS-CoV-2.

Antiviral agents are being developed to treat viral infections. For example, β-D-N(4)-hydroxycytidine (NHC, 1-((2R,3R,4S,5R)-3,4-dihydroxy-5-(hydroxymethyl)tetrahydrofuran-2-yl)-4-(hydroxyamino)pyrimidin-2 (1H)-one),

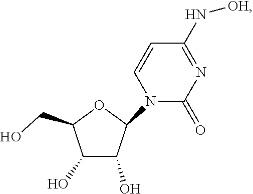 [SMILES: O=c1nc(NO)ccn1[C@@H]1O[C@H](CO)[C@@H](O)[C@H]1O]

has antipestivirus and antihepacivirus activities. ANTIMICROB AGENTS CHEMOTHER, 2003, 47 (1): 244-54. While NHC has been shown to have limited oral bioavailability in non-human primates (see Toots et al., SCI. TRANSL. MED. 11, eaax5866 (2019)), prodrugs of NHC, including {(2R,3S,4R,5R)-3,4-dihydroxy-5-[4-(hydroxyimino)-2-oxo-3,4-dihydropyrimidin-1 (2H)-yl]oxolan-2-yl}methyl 2-methylpropanoate):

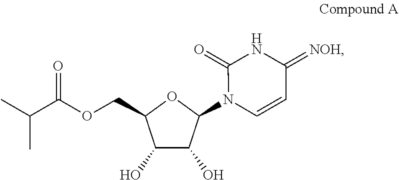

which is also known as uridine 4-oxime 5′-(2-methylpropanoate) and molnupiravir, are orally bioavailable and are understood to be potentially useful for the treatment of viral infections caused by at least Eastern Equine Encephalitis Virus (EEEV), Western Equine Encephalitis Virus (WEEV), and Venezuelan Equine Encephalitis Virus (VEEV), Chikungunya fever virus (CHIK), Ebola virus, influenza virus, respiratory syncytial virus (RSV), Zika virus, and coronaviruses, such as Severe Acute Respiratory Syndrome Coronavirus (SARS-CoV), Middle East Respiratory Syndrome Coronavirus (MERS-CoV), and, most recently, SARS-CoV-2 (also known as 2019-nCoV).

β-D-N(4)-hydroxycytidine, its prodrugs, its derivatives, and methods for making the same are illustrated in PCT International Patent Application No. PCT/US2015/066144, which published as PCT International Patent Application Publication No. WO2016/106050, and U.S. patent application Ser. No. 15/537,087, which published as United States Patent Application Publication No. US2019/0022116, and U.S. patent application Ser. No. 16/921,359, each of which are incorporated herein by reference in their entirety. Compound A, and pharmaceutically acceptable salts, tautomers, and prodrugs thereof, as well as additional derivatives of β-D-N(4)-hydroxycytidine, and methods for making the same are illustrated in PCT International Patent Application No. PCT/US2018/064503, which published as PCT International Patent Application Publication No. WO2019/113462, and U.S. patent application Ser. No. 16/755,779, now U.S. Pat. No. 11,331,331, and U.S. patent application Ser. No. 17/465,344, which published as US 2022/0016153 A1, and in U.S. Provisional Patent Application No. 63/127,484, each of which are incorporated herein by reference in their entirety.

Compound A is being developed for the treatment of viral infections caused by SARS-CoV-2 and other pathogens, as well as for the treatment of COVID-19 caused by SARS-CoV-2. However, the 800 mg dose, administered twice daily for five days, takes the form of four size 0 capsules that are 21.7 mm×7.6 mm and have a 200 mg unit dosage strength, which creates a significant pill burden, both in terms of the size of each capsule and in terms of the number of capsules administered at each dosage interval. Some patient populations, such as pediatric patients, elderly patients, and other patient populations having specific medical limitations, may find these capsules difficult to swallow. In addition, the 800 mg dosage also cannot be adjusted for pediatric patients or for other medical reasons, beyond adjusting the number of size 0 capsules having 200 mg unit dosage strength.

The goal of any drug delivery system is to provide a therapeutic amount of drug to the proper site in the body to achieve, and then maintain the desired drug concentration. The most convenient and commonly employed route of drug delivery has historically been by solid oral dosage forms, particularly tablets, and capsules. However, difficulty swallowing tablets and capsules is a problem for many patients, including pediatric and elderly patients, and can lead to a variety of adverse events, and patient noncompliance with treatment regimens. In addition, conventional tablets and capsules are limited by their rigid dose content.

There remains a need for novel antiviral oral dosage forms that are more amenable to patient populations that are averse to standard oral dosage forms and provide the ability to dose patient populations that cannot swallow intact conventional tablet or capsule formulations. There is also a need for antiviral oral dosage forms that allow for dose adjustment for specific patient populations.

### SUMMARY

The present disclosure relates to oral dosage forms of pharmaceutical formulations that comprise an antiviral nucleoside, one or more compression aids, one or more glidants, and one or more lubricants, and one or more disintegrants, wherein the oral dosage form is a pellet having a diameter of less than or equal to 4.0 mm. The antiviral nucleoside is selected from prodrugs of β-D-N(4)-hydroxycytidine,

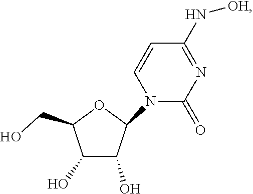 [SMILES: O=c1nc(NO)ccn1[C@@H]1O[C@H](CO)[C@@H](O)[C@H]1O]

such as {(2R,3S,4R,5R)-3,4-dihydroxy-5-[4-(hydroxyimino)-2-oxo-3,4-dihydropyrimidin-1 (2H)-yl] oxolan-2-yl}methyl 2-methylpropanoate):

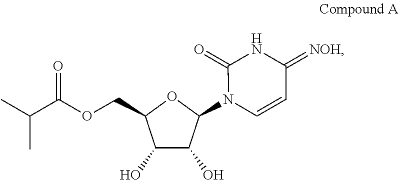

or a pharmaceutically acceptable salt, tautomer, or prodrug thereof.

Other embodiments, aspects and features of the present invention are either further described in or will be apparent from the ensuing description, examples, and appended claims. The summary of the technology described above is non-limiting and other features and advantages of the technology will be apparent from the following detailed description, and from the claims.

### DETAILED DESCRIPTION

### Definitions

Certain technical and scientific terms are specifically defined below. Unless specifically defined elsewhere in this document, all other technical and scientific terms used herein have the meaning commonly understood by one of ordinary skill in the art to which this disclosure relates.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, including the appended claims, the singular forms of words such as “a,” “an,” and “the,” include their corresponding plural references unless the context clearly dictates otherwise.

The terms “administration of” and or “administering” a compound should be understood to include providing a compound described herein, or a pharmaceutically acceptable salt thereof, and compositions of the foregoing to a subject.

As used herein, the terms “at least one” item or “one or more” item each include a single item selected from the list as well as mixtures of two or more items selected from the list.

The term “subject” (alternatively “patient” or “participant”, as in a clinical trial participant) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorpha (e.g., rabbits), rodents (e.g., rats or mice), and procyon lotor (e.g., raccoons). In particular embodiments, the subject is human. In some embodiments, the subject is an adult patient. In other embodiments, the subject is a pediatric patient. Those “in need of treatment” include those subjects that may benefit from treatment with the formulations of the invention, e.g., a patient suffering from a viral infection, such as infection by SARS-CoV-2.

The term “subject in need thereof” as used herein refers to a subject diagnosed with, or suspected of having, a viral infection, such as infection by SARS-CoV-2 (either symptomatic or asymptomatic); a subject at risk of being exposed to a viral infection, such as at risk of being exposed to a viral infection, such as infection by SARS-CoV-2 (such as, for example, health care workers who may be at risk of exposure to SARS-CoV-2); a subject exposed to a viral infection, such as infection by SARS-CoV-2 (such as household contacts of COVID-19 patients or asymptomatic patients infected with SARS-CoV-2), as defined herein.

As used herein, the term “COVID-19” refers to the disease caused by SARS-CoV-2 infection. Subjects infected with SARS-CoV-2 who have developed symptoms are considered to have COVID-19.

Certain subjects may be considered to be at increased risk for severe illness from COVID-19. Such individuals may have one or more underlying medical conditions associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding individuals on dialysis or who have reduced estimated glomerular filtration rate (eGFR)<30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

As used herein, the terms “prophylaxis” and “antiviral prophylaxis” refer to all processes intended to prevent disease. Prophylaxis may occur prior to exposure to a viral infection, such as infection by SARS-CoV-2 (pre-exposure, for example, in health care workers who may be exposed to such infection) or after a potential exposure to a viral infection, such as infection by SARS-CoV-2 (post-exposure, for example, in household members or caregivers of symptomatic or asymptomatic patients infected with SARS-CoV-2).

The term “simultaneous administration” as used herein refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

The term “tablet” as used herein is intended to encompass compressed pharmaceutical dosage formulations of all shapes and sizes. Tablets that have a reduced size relative to conventional tablet and capsule formulations may be referred to herein alternatively as “pellet(s)”, “granule(s)”, or “mini-tablet(s)”. Pellets may have a diameter range of from about 1.00 mm to about 4.00 mm.

Unless expressly stated to the contrary, all ranges cited herein are inclusive; the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of ±1.00%, ±2.00%, ±3.00%, ±4.00%, ±5.00%, and ±10.0% and their numerical equivalents. “About” when used to modify a numerically defined parameter (e.g., the dose of an antiviral nucleoside, or the length of treatment time with a combination therapy described herein) means that the parameter may vary by as much as 10.0% below or above the stated numerical value for that parameter; where appropriate, the stated parameter may be rounded to the nearest whole number. For example, a dose of about 5.00 mg/kg may vary between 4.50 mg/kg and 5.50 mg/kg. In addition, the term “or,” as used herein, denotes alternatives that may, where appropriate, be combined; that is, the term “or” includes each listed alternative separately as well as their combination.

Unless otherwise defined, all technical and scientific terms used herein have the same meaning as commonly understood by one of ordinary skill in the art to which this disclosure relates. In case of conflict, the present specification, including definitions, will control.

Throughout this specification and claims, the word “comprise,” or variations such as “comprises” or “comprising” are used in an inclusive sense, i.e., to specify the presence of the stated features but not to preclude the presence or addition of further features that may materially enhance the operation or utility of any of the embodiments of the invention, unless the context requires otherwise due to express language or necessary implication. Unless otherwise required by context, singular terms shall include pluralities and plural terms shall include the singular. Any example(s) following the term “e.g.” or “for example” is not meant to be exhaustive or limiting.

It is understood that wherever embodiments are described herein with the language “comprising,” otherwise analogous embodiments described in terms of “consisting of” and/or “consisting essentially of” are also provided.

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

The term “pharmaceutically effective amount” or “effective amount” means an amount whereby sufficient therapeutic composition or formulation is introduced to a patient to treat a disease or condition. One skilled in the art recognizes that this level may vary according to the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.100%, 0.500%, 1.00%, 2.00%, 3.00%, 4.00%, 5.00%, or 10.0%.

“Pharmaceutically acceptable” refers to excipients (vehicles, additives) and compositions that can reasonably be administered to a subject to provide an effective dose of the active ingredient employed and that are “generally regarded as safe”, e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the United States Pharmacopeia or another generally recognized pharmacopeia for use in animals, and more particularly in humans.

The term “pharmaceutically acceptable carrier” refers to any inactive substance that is suitable for use in a formulation for the delivery of a therapeutic agent. A carrier may be an anti-adherent, binder, coating, disintegrant, filler or diluent, lubricant, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

The term “pharmaceutical formulation” refers to preparations that are in such form as to permit the active ingredients to be effective. The term “formulation” and “pharmaceutical formulation” are used interchangeably throughout.

Direct compression is defined as a process involving pre-milling each ingredient of a formulation (e.g., the active drug substance and excipients) into a powder, mixing the powdered ingredients and any lubricants, and compressing the mixed powders into tablets. When the formulation is to be processed by direct compression, the active ingredient, herein an antiviral compound, may be provided for pre-milling either directly, “as is”, from synthesis or after purification by a suitable method, such as recrystallization.

Wet granulation is defined as a process involving granulating a powder with liquid (aqueous, non-aqueous, hot melt, etc.) to achieve the desired properties for subsequent downstream processes. The liquid (e.g., water, binder solution) is added to a powder blend while the powder blend is being continuously mixing, leading to granule nucleation and growth. When the formulation is to be processed by wet granulation, the active ingredient, herein an antiviral compound, may be provided directly, “as is”, from synthesis or after purification by a suitable method, such as recrystallization.

Roller compaction is defined as an agglomeration process where a powder is compressed into a dense compact (or ribbon) using two counter-rotating rollers. The pressing forces from the rollers cause the particles to deform plastically to achieve the dense compact. The dense compact is subsequently broken up and milled into granules. The advantages of roller compaction include improved flow, better content uniformity, and/or less sticking. Roller compaction is also preferred for moisture sensitive and/or heat sensitive formulations.

Excipients that are mixed with other ingredients (including the active ingredient) prior to granulation, and are thus incorporated within the granules, comprise the intragranular component of the formulation. Excipients that are mixed with the dry granules before the complete mixture is compacted comprise the extragranular component of the formulation. In certain embodiments, individual excipients may be incorporated as intragranular components, as extragranular components, or as both intragranular and extragranular components.

The term “compression aid” refers to any substance used in the preparation of pharmaceutical dosage forms, such as pellets, that is a pharmacologically inactive substance that may be compacted easily even when mixed with an active drug substance, and that improves flowability and compressibility during manufacture of pellets by direct compression. Examples of suitable pharmaceutically acceptable compression aids for use in the oral dosage forms described herein include directly compressible starches, dibasic calcium phosphate, spray-dried lactose, anhydrous lactose, spray crystallized maltose, crystalline sorbitol, mannitol, microfine cellulose, and microcrystalline cellulose.

The term “glidant” refers to any substance used in the preparation of pharmaceutical dosage forms, such as pellets, that enhances the flow of a granular mixture by reducing friction between the particles. Examples of suitable pharmaceutically acceptable glidants for use in the oral dosage forms described herein include corn starch, talc, colloidal silica, and tri-basic calcium phosphate (Ca₅(PO₄)₃(OH)).

The term “disintegrant” refers to any substance used in the preparation of pharmaceutical dosage forms, such as pellets, that causes them to disintegrate and release their medicinal substances on contact with moisture. In general, disintegrants and super-disintegrants absorb water and expand when wet, causing tablet formulations to break apart in the digestive tract, releasing the active ingredient(s) for absorption. Super-disintegrants have a greater effectiveness at low concentrations (when compared with disintegrants) in tablet or pellet formulations. In some instances, the disintegrant or super-disintegrant can react with the active ingredient. Examples of suitable pharmaceutically acceptable disintegrants for use in the oral dosage forms described herein include calcium alginate, calcium sodium alginate, calcium carboxymethylcellulose, calcium cellulose glycolate, carmellose calcium, microcrystalline cellulose, powdered cellulose, chitosan hydrochloride, corn starch, pregelatinized starch, crospovidone, low-substituted hydroxypropyl cellulose, hydroxypropyl starch, magnesium aluminum silicate, methylcellulose, sodium alginate, starch, sodium starch glycolate, and croscarmellose sodium.

The term “lubricant” refers to any substance used in the preparation of pharmaceutical dosage forms, such as pellets, that decreases friction between the surfaces of the compression apparatus and the pellet itself. Examples of suitable pharmaceutically acceptable lubricants for use in the oral dosage forms described herein include magnesium silicate, calcium stearate, stearic acid, talc, sodium lauryl sulfate, magnesium lauryl sulfate, and magnesium stearate.

The terms “binder” and/or “filler” as used herein refer to excipients that are incorporated into the pharmaceutical formulation to ensure that the final form will have the required mechanical strength. Binders suitable for use in the pharmaceutical compositions provided herein include, but are not limited to, starches, cellulose and its derivatives (e.g., ethylcellulose, cellulose acetate, carboxymethyl cellulose calcium, sodium carboxymethyl cellulose, methylcellulose, hydroxypropyl methylcellulose, hydroxypropyl cellulose), polyvinyl pyrrolidone, and mixtures thereof. Examples of fillers suitable for use in the pharmaceutical compositions provided herein include, but are not limited to, microcrystalline cellulose, powdered cellulose, mannitol, lactose, calcium phosphate, starch, pre-gelatinized starch, and mixtures thereof.

A “stable” formulation is one in which the antiviral compound therein essentially retains its physical stability and/or chemical stability upon storage. Stability can be measured at a selected temperature and relative humidity for a selected time period. For example, in one embodiment, a stable formulation is a formulation with no significant changes observed at ambient temperature (25.0° C. to 30.0° C.) and relative humidity below 75.0% for at least 12 months. In another embodiment, a stable formulation is a formulation with no significant changes observed at ambient temperature (25.0° C. to 30.0° C.) and relative humidity below 75.0% for at least 18 months. Potency is typically within 90.0-110% of the target potency value. Additional abbreviations may be defined throughout this disclosure.

### Oral Dosage Forms

The present disclosure relates to oral dosage forms of pharmaceutical formulations that comprise an antiviral nucleoside, one or more compression aids, one or more glidants, and one or more lubricants, and one or more disintegrants, wherein the oral dosage form is a pellet having a diameter of less than or equal to 4.0 mm.

In embodiments, the antiviral nucleoside is selected from prodrugs of β-D-N(4)-hydroxycytidine,

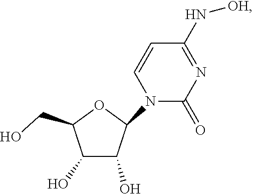 [SMILES: O=c1nc(NO)ccn1[C@@H]1O[C@H](CO)[C@@H](O)[C@H]1O]

In specific embodiments, the antiviral nucleoside is selected from the antiviral nucleosides as disclosed in PCT International Patent Application No. PCT/US2015/066144, which published as WO2016/106050, PCT International Application No. PCT/US2017/021759, which published as WO2017/156380, and PCT International Patent Application No. PCT/US2018/064503, which published as PCT International Patent Application Publication No. WO2019/113462, which are incorporated herein by reference in their entirety. In more specific embodiments, the antiviral nucleoside is {(2R,3S,4R,5R)-3,4-dihydroxy-5-[4-(hydroxyimino)-2-oxo-3,4-dihydropyrimidin-1 (2H)-yl] oxolan-2-yl}methyl 2-methylpropanoate):

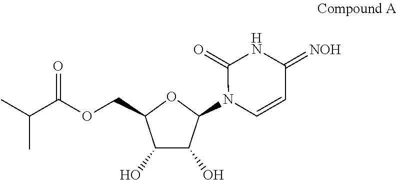

or a pharmaceutically acceptable salt, tautomer, or prodrug thereof. In more specific embodiments, the antiviral nucleoside is Compound A. In other specific embodiments, the antiviral nucleoside is a prodrug of Compound A.

Methods of preparing NHC and prodrugs thereof are illustrated in PCT International Patent Application No. PCT/US2015/066144, which published as PCT International Patent Application Publication No. WO2016/106050, and U.S. patent application Ser. No. 15/537,087, which published as United States Patent Application Publication No. US2019/0022116, and U.S. patent application Ser. No. 16/921,359, each of which are incorporated herein by reference in their entirety. Methods of preparing additional prodrugs of NHC, including Compound A, and tautomers, prodrugs, and derivatives thereof, are disclosed in PCT International Patent Application No. PCT/US2018/064503, which published as PCT International Patent Application Publication No. WO2019/113462, and U.S. patent application Ser. No. 16/755,779, now U.S. Pat. No. 11,331,331, and U.S. patent application Ser. No. 17/465,344, which published as US 2022/0016153 A1, and in U.S. Provisional Patent Application No. 63/127,484, which are incorporated herein by reference in their entirety.

In a first embodiment, the oral dosage form is a pellet having a diameter in a range of from about 1.00 mm to about 4.00 mm, such as from about 1.00 mm to about 2.80 mm, from about 1.80 mm to about 2.20 mm, or about 2.00 mm.

Conventional oral dosage forms may not be appropriate for use in treating or preventing viral infections in all subjects. Oral dosage forms that have a reduced size relative to conventional tablet and capsule formulations are desirable for many applications, including pediatric dosage, for patients with dysphagia (swallowing difficulties), and in applications where rapid and/or flexible dose adjustments are needed. Pellets having a diameter that is equal to or less than 4 mm may be desirable in such applications. For example, the oral dosage forms described herein may be used to provide incremental changes to pediatric dosages by changing the number of pellets administered, or a desired dose may be provided as pellets, rather than as a conventional capsule or tablet, for ease of swallowing. Such pellets can be produced relatively simply, with uniform size and consistent active substance content. Very precise individual division of the dose could be carried out by counting pellets.

Because the oral dosage forms described herein have the dimensions of the first embodiment, the size of the pellets allows them to be swallowed more easily by young children, as well as geriatric patients who may have difficulty swallowing. For pediatric patients, the oral dosage forms described herein allow for the use of weight-based dosing to children as young as six months to two years of age. In addition, the oral dosage forms described herein can easily be mixed with liquid and soft foods to assist in administration to younger pediatric patients, and the oral dosage forms described herein can be dissolved in water (or other suitable liquid) and administered to infants, including neonates and preterm neonates.

The oral dosage forms described herein can be administered as multiple unit dosage forms, allowing dose adjustment, and are often easier for patients to swallow, due to their reduced size. The oral dosage forms, the pellet formulations described herein, are more appropriate for pediatric and geriatric patients than traditional capsules or tablets, because they offer better swallowability and dose flexibility.

Furthermore, the small size of pellets of these formulations, facilitates fast disintegration time and allows dispersion in a liquid prior to administration, which may be necessary or desirable for younger pediatric patients as well as patients who are unable to swallow traditional tablet or capsule dosage forms. Once the pellets are dispersed in a liquid, the liquid may be administered by swallowing or by nasogastric tube as needed.

For at least these reasons, oral dosage forms, the pellet formulations described herein, are particularly useful for administering to pediatric, and elderly, patients, as they allow for ease of swallowing and can be easily dissolved and dispersed in liquids. Thus, such formulations offer several additional advantages over standard tablets and capsules.

In embodiments, the disclosure provides an oral dose comprising from about 1 to about 400 pellets as provided herein. In further aspects of this embodiment, the oral dose comprises from about 1 to about 400 pellets provided in a sachet. In still further aspects of this embodiment, the oral dosage form is provided as from about 1 to about 400 pellets in a capsule.

In a second embodiment, the oral dosage form is an uncoated pellet having a weight in a range of from about 1.00 mg to about 10.0 mg, such as from about 6.00 mg to about 7.00 mg, from about 6.25 mg to about 6.75 mg, or about 6.50 mg.

In a third embodiment, the oral dosage form is a pellet comprising the antiviral nucleoside in an amount of from about 0.0650 mg to about 5.20 mg, such as from about 1.30 mg to about 4.60 mg, from about 1.80 mg to about 2.20 mg, or about 2.00 mg.

In a fourth embodiment, the oral dosage form is a pellet comprising the antiviral nucleoside in an amount relative to the total weight of the tablet of from about 10.0 wt % to about 80.0 wt %, such as from about 20.0 wt % to about 40.0 wt %, from about 20.8 wt % to about 40.8 wt %, or about 30.8 wt %.

In a fifth embodiment, the one or more compression aid is selected from the group consisting of directly compressible starches, dibasic calcium phosphate, spray-dried lactose, anhydrous lactose, spray crystallized maltose, spray crystallized dextrose, crystalline sorbitol, mannitol, sucrose, microfine cellulose, and microcrystalline cellulose. In aspects of this embodiment, the one or more compression aid is selected from the group consisting of directly compressible starches, dibasic calcium phosphate, spray-dried lactose, anhydrous lactose, mannitol, microfine cellulose, and microcrystalline cellulose. In specific aspects of this embodiment, the one or more compression aid is microcrystalline cellulose. In a first instance, the one or more compression aid is present in an amount of from about 0.195 mg to about 6.00 mg, such as from about 0.90 mg to about 5.12 mg, from about 2.71 mg to about 4.52 mg, or about 3.92 mg. In a second instance, the one or more compression aid is present in an amount relative to the total weight of the pellet of from about 3.00 wt % to about 92.3 wt %, such as from about 13.9 wt % to about 78.8 wt %, from about 41.7 wt % to about 69.5 wt %, or about 60.2 wt %.

In a sixth embodiment, the one or more glidant is selected from the group consisting of starch, corn starch, talc, silica, colloidal silica, and tri-basic calcium phosphate. In aspects of this embodiment, the one or more glidant is selected from the group consisting of starch, talc, and tri-basic calcium phosphate. In specific aspects of this embodiment, the one or more glidant is tri-basic calcium phosphate. In a first instance, the one or more glidant is present in an amount of from about 0.016 mg to about 0.195 mg, such as from about 0.0325 mg to about 0.130 mg, from about 0.0488 mg to about 0.975 mg, or about 0.0650 mg. In a second instance, the one or more glidant is present in an amount relative to the total weight of the pellet of from about 0.250 wt % to about 3.00 wt %, such as from about 0.500 wt % to about 2.00 wt %, from about 0.750 wt % to about 1.50 wt %, or about 1.00 wt %.

In a seventh embodiment, the one or more lubricant is selected from the group consisting of magnesium silicate, calcium stearate, stearic acid, talc, sodium lauryl sulfate, magnesium lauryl sulfate, sodium stearyl fumarate, and magnesium stearate. In aspects of this embodiment, the one or more lubricant is magnesium stearate. In specific aspects of this embodiment, the one or more lubricant is extragranular magnesium stearate. In a first instance, the one or more lubricant is present in an amount of from about 0.0325 mg to about 0.3250 mg, such as from about 0.0650 mg to about 0.260 mg, from about 0.114 mg to about 0.146 mg, or about 0.130 mg. In a second instance, the one or more lubricant is present in an amount relative to the total weight of the pellet of from about 0.500 wt % to about 5.00 wt %, such as from about 1.00 wt % to about 4.00 wt %, from about 1.75 wt % to about 2.25 wt %, or about 2.00 wt %.

In an eighth embodiment, the one or more disintegrant is selected from the group consisting of calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, hydroxypropyl cellulose, low-substituted hydroxypropyl cellulose, hydroxypropyl starch, magnesium aluminum silicate, methylcellulose, sodium alginate, starch, sodium starch glycolate, and croscarmellose sodium. In aspects of this embodiment, the one or more disintegrant is selected from the group consisting of calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, low-substituted hydroxypropyl cellulose, hydroxypropyl starch, magnesium aluminum silicate, methylcellulose, sodium alginate, starch, sodium starch glycolate, and croscarmellose sodium. In specific aspects of this embodiment, the one or more disintegrant is croscarmellose sodium. In a first instance, the one or more disintegrant is present in an amount of from about 0.0163 mg to about 0.585 mg, such as from about 0.065 mg to about 0.488 mg, from about 0.195 mg to about 0.423 mg, or about 0.390 mg. In a second instance, the one or more glidant is present in an amount relative to the total weight of the pellet of from about 0.250 wt % to about 9.00 wt %, such as from about 1.00 wt % to about 7.50 wt %, from about 3.00 wt % to about 6.50 wt %, or about 6.00 wt %.

In a ninth embodiment, the oral dosage form further comprises one or more binders. In aspects of this embodiment, the one or more binders is selected from the group consisting of starches, cellulose and its derivatives (e.g., ethylcellulose, cellulose acetate, carboxymethyl cellulose calcium, sodium carboxymethyl cellulose, methylcellulose, hydroxypropyl methylcellulose, hydroxypropyl cellulose), polyvinyl pyrrolidone, and mixtures thereof, and fillers such as microcrystalline cellulose, powdered cellulose, mannitol, lactose, calcium phosphate, starch, pre-gelatinized starch, and mixtures thereof. In aspects of this embodiment, the one or more binders is selected from the group consisting of starches, cellulose, ethylcellulose, cellulose acetate, carboxymethyl cellulose calcium, sodium carboxymethyl cellulose, methylcellulose, hydroxypropyl methylcellulose, hydroxypropyl cellulose, polyvinyl pyrrolidone, microcrystalline cellulose, powdered cellulose, mannitol, lactose, calcium phosphate, starch, pre-gelatinized starch, and mixtures thereof. In specific aspects of this embodiment, the one or more binders is hydroxypropyl cellulose. In a first instance, the one or more binder is present in an amount of from about 0.000 mg to about 0.3900 mg, such as from about 0.0975 mg to about 0.2925 mg, from about 0.1625 mg to about 0.2275 mg, or about 0.1950 mg. In a second instance, one or more binder is present in an amount relative to the total weight of the pellet of from about 0.000 wt % to about 6.000 wt %, such as from about 1.500 wt % to about 4.500 wt %, from about 2.500 wt % to about 3.500 wt %, or about 3.000 wt %.

In a tenth embodiment, the oral dosage form is a pellet optionally further comprising a coating. In aspects, the coating may comprise hydroxypropylmethylcellulose, hydroxypropylcellulose, titanium dioxide, calcium carbonate, polyvinyl acetate, talc, sweeteners, and colorants.

Embodiments of this disclosure are directed to oral dosage forms of pharmaceutical formulations that comprise
an antiviral nucleoside selected from prodrugs of β-D-N(4)-hydroxycytidine,

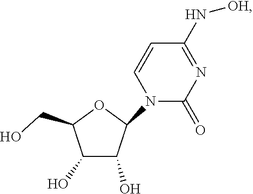 [SMILES: O=c1nc(NO)ccn1[C@@H]1O[C@H](CO)[C@@H](O)[C@H]1O]

one or more compression aids selected from the group consisting of directly compressible starches, dibasic calcium phosphate, spray-dried lactose, anhydrous lactose, spray crystallized maltose, spray crystallized dextrose, crystalline sorbitol, mannitol, sucrose, microfine cellulose, and microcrystalline cellulose,
one or more glidants selected from the group consisting of starch, corn starch, talc, silica, colloidal silica, and tri-basic calcium phosphate,
one or more lubricants selected from the group consisting of magnesium silicate, calcium stearate, stearic acid, talc, sodium lauryl sulfate, magnesium lauryl sulfate, sodium stearyl fumarate, and magnesium stearate, and
one or more disintegrants selected from the group consisting of calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, hydroxypropyl cellulose, low-substituted hydroxypropyl cellulose, hydroxypropyl starch, magnesium aluminum silicate, methylcellulose, sodium alginate, starch, sodium starch glycolate, and croscarmellose sodium,
   wherein the oral dosage form is a pellet having a diameter of less than or equal to 4.0 mm.

Certain embodiments of this disclosure are directed to oral dosage forms of pharmaceutical formulations that comprise
an antiviral nucleoside that is {(2R,3S,4R,5R)-3,4-dihydroxy-5-[4-(hydroxyimino)-2-oxo-3,4-dihydropyrimidin-1 (2H)-yl] oxolan-2-yl}methyl 2-methylpropanoate):

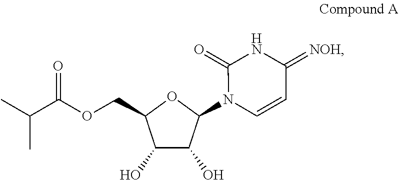

or a pharmaceutically acceptable salt, tautomer, or prodrug thereof,
one or more compression aids selected from the group consisting of directly compressible starches, dibasic calcium phosphate, spray-dried lactose, anhydrous lactose, mannitol, microfine cellulose, and microcrystalline cellulose,
one or more glidants selected from the group consisting of starch, talc, silica, and tri-basic calcium phosphate,
one or more lubricants that is magnesium stearate, and
one or more disintegrants selected from the group consisting of calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, low-substituted hydroxypropyl cellulose, hydroxypropyl starch, magnesium aluminum silicate, methylcellulose, sodium alginate, starch, sodium starch glycolate, and croscarmellose sodium,
   wherein the oral dosage form is a pellet having a diameter of less than or equal to 4.0 mm.

Certain specific embodiments of this disclosure are directed to oral dosage forms of pharmaceutical formulations that comprise
{(2R,3S,4R,5R)-3,4-dihydroxy-5-[4-(hydroxyimino)-2-oxo-3,4-dihydropyrimidin-1 (2H)-yl] oxolan-2-yl}methyl 2-methylpropanoate):

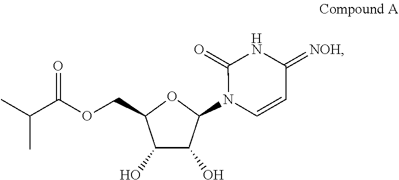

or a pharmaceutically acceptable salt, tautomer, or prodrug thereof,
microcrystalline cellulose,
tri-basic calcium phosphate,
magnesium stearate, and
croscarmellose sodium,
   wherein the oral dosage form is a pellet having a diameter of less than or equal to 4.0 mm.

With respect to manufacturing, oral dosage forms as described herein possess robust mechanical properties due to the combination of Compound A and excipients, as described in the embodiments above and the combination of these embodiments. The oral dosage forms can be coated reproducibly and require less coating material, due to their constant specific surface area, smooth outer surface, and robust mechanical properties. See Munday, D. L., 1994. Drug Dev. Ind. Pharm. 20, 2369-2379. Thus, such formulations offer several additional advantages over standard tablets and capsules.

### Treatment or Prevention of Viral Infection

Antiviral agents, including NHC and prodrugs of NHC, such as Compound A, are known to be useful in treating viral infection and may be useful in preventing viral infection. Oral dosage forms, such as pellets, as described herein may be useful in treating viral infection and in preventing viral infection in applications, such as in dosing pediatric patients, dosing patients with dysphagia, and when rapid and/or flexible dose adjustments are needed to appropriately treat a patient.

The present disclosure relates to methods of treating a viral infection, wherein the method comprises administering to a subject in need thereof oral dosage forms as described herein; wherein the viral infection is an infection by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In a second aspect of these embodiments, the virus is selected from the group consisting of RSV, influenza A virus, influenza B virus, filoviridae, human coronavirus, SARS-CoV-1, MERS-CoV, SARS-CoV-2, and Ebola virus. In specific aspects, the virus is selected from the group consisting of influenza A virus and influenza B virus. In further specific aspects, the virus is selected from the group consisting of human coronavirus, SARS-CoV-1, MERS-CoV, and SARS-CoV-2. In specific embodiments, the virus is SARS-CoV-2.

In embodiments of the treatment methods disclosed herein, the oral dosage forms are administered once daily, as a single dose. In particular aspects of these embodiments, the single daily dose may be provided as 1 to 400 2 mg individual pellets.

In embodiments of the treatment methods disclosed herein, the oral dosage forms are administered twice daily, as two individual doses. In particular aspects of these embodiments, each individual dose may be provided as 1 to 400 2 mg individual pellets.

In embodiments of the treatment methods disclosed herein, said method comprises administering to a subject in need thereof oral dosage forms as described herein; wherein the oral dosage forms are administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered once daily for 3 to 6 days, such as for 3 days, 4 days, 5 days, or 6 days. In another specific embodiment, the oral dosage forms are administered once daily for 5 days.

In embodiments of the treatment methods disclosed herein, said method comprises administering to a subject in need thereof oral dosage forms as described herein; wherein the oral dosage forms are administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered twice daily for 3 to 6 days, such as for 3 days, 4 days, 5 days, or 6 days. In another specific embodiment, the oral dosage forms are administered twice daily for 5 days.

The disclosure further relates to a method of treating a viral infection, said method comprising administering to a subject in need thereof oral dosage forms as described herein; wherein the therapy commences from 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the oral dosage forms are administered beginning less than 5 days after onset of symptoms, such as less than 1 day, 2 days, 3 days, 4 days, or 5 days after onset of symptoms of viral infection. Symptoms of viral infection may include one or more of cough, sore throat, nasal congestion, runny nose, shortness of breath or difficulty breathing, muscle or body aches, fatigue/tiredness, feeling hot/feverish, chills, headache, nausea, vomiting, and diarrhea, although symptoms may vary by the type and severity of the viral infection. For example, symptoms of COVID-19 caused by SARS-CoV-2 viral infection may include one or more of cough, sore throat, nasal congestion, runny nose, shortness of breath or difficulty breathing, muscle or body aches, fatigue/tiredness, feeling hot or feverish, chills, headache, nausea, vomiting, diarrhea, loss of taste, and loss of smell.

The disclosure further relates to the above-described methods of treating a viral infection, wherein said subjects may be considered or is determined to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to the above-described methods of treating a viral infection, wherein said method reduces the risk of hospitalization or death for the subject. In embodiments, the method may result in a reduction in risk of hospitalization or death for the subject. In specific embodiments, the method may result in a reduction in risk of hospitalization or death for the subject of about 1 to about 10 percent, such as from about 5 to about 7.5 percent, or about 6.8 percent. In further embodiments, the method may result in a relative reduction in risk of hospitalization or death for the subject of up to about 50 percent.

Additionally, present disclosure relates to the above-described methods of providing antiviral prophylaxis, wherein the viral infection is caused by virus is selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of the prophylaxis methods disclosed herein, the oral dosage forms are administered once daily, as a single dose. In particular aspects of these embodiments, the single daily dose may be provided as 1 to 400 2 mg individual pellets.

In embodiments of the prophylaxis methods disclosed herein, the oral dosage forms are administered twice daily, as two individual doses. In particular aspects of these embodiments, each individual dose may be provided as 1 to 400 2 mg individual pellets.

The disclosure further relates to methods of providing antiviral prophylaxis, said method comprising administering to a subject in need thereof oral dosage forms as described herein; wherein the oral dosage forms are administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days.

The disclosure further relates to methods of providing antiviral prophylaxis, said method comprising administering to a subject in need thereof oral dosage forms as described herein; wherein the oral dosage forms are administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days.

The disclosure further relates to methods of providing antiviral prophylaxis, said method comprising administering to a subject in need thereof oral dosage forms as described herein; wherein the administration commences prior to exposure to a viral infection or after a potential exposure to a viral infection. In specific embodiments, the oral dosage forms are administered beginning prior to potential exposure to viral infection. In other specific embodiments, the oral dosage forms are administered from 1 to 10 days after potential exposure to a viral infection, such as 1 day, 2 days, 3 days, 4 days, 5 days, 6 days, 7 days, 8 days, 9 days, or 10 days after potential exposure. In specific embodiments, the oral dosage forms are administered beginning less than 5 days after potential exposure, such as less than 1 day, 2 days, 3 days, 4 days, or 5 days after potential exposure.

The disclosure further relates to the above-described methods of providing antiviral prophylaxis, wherein said subjects may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure relates to prophylaxis methods as described above, wherein said method reduces the risk of hospitalization or death for the subject. In embodiments, the method may result in a reduction in risk of hospitalization or death for the subject. In specific embodiments, the method may result in a reduction in risk of hospitalization or death for the subject of about 1 to about 10 percent, such as from about 5 to about 7.5 percent, or about 6.8 percent. In further embodiments, the method may result in a relative reduction in risk of hospitalization or death for the subject of up to about 50 percent.

Embodiments provided by this disclosure also include oral dosage forms as described herein for use as a medicament for the treatment of viral infection. In specific embodiments, the viral infection is an infection by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

The disclosure also provides oral dosage forms as described herein for use as a medicament for the treatment of viral infection, where the patient has previously (e.g., within 24 hours) been treated with another agent, which may be as described above.

In embodiments, the oral dosage forms as described herein for use as a medicament for the treatment of viral infection are administered once daily, as a single dose. In particular aspects of these embodiments, the single daily dose may be provided as 1 to 400 2 mg individual pellets.

In embodiments, the oral dosage forms as described herein for use as a medicament for the treatment of viral infection are administered twice daily, as two individual doses. In particular aspects of these embodiments, each individual dose may be provided as 1 to 400 2 mg individual pellets.

In embodiments of oral dosage forms for use as a medicament for the treatment of viral infection, the oral dosage forms are administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered once daily for 3 to 6 days, such as for 3 days, 4 days, 5 days, or 6 days. In another specific embodiment, the oral dosage forms are administered once daily for 5 days.

In embodiments of oral dosage forms for use as a medicament for the treatment of viral infection, wherein the oral dosage forms are administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered twice daily for 3 to 6 days, such as for 3 days, 4 days, 5 days, or 6 days. In another specific embodiment, the oral dosage forms are administered twice daily for 5 days.

The disclosure further relates to oral dosage forms for use as a medicament for the treatment of viral infection, wherein administration of the oral dosage forms is commenced 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the oral dosage forms are administered beginning less than 5 days after onset of symptoms, such as less than 1 day, 2 days, 3 days, 4 days, or 5 days after onset of symptoms of viral infection. Symptoms of viral infection may be as described above.

The disclosure further relates to oral dosage forms for use as a medicament for the treatment of viral infection, wherein the pellets are administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to oral dosage forms for use as a medicament for the treatment of viral infection, wherein administration of oral dosage forms reduces the risk of hospitalization or death for the subject. In embodiments, administration of oral dosage forms may result in a reduction in risk of hospitalization or death for the subject. In specific embodiments, administration of oral dosage forms may result in a reduction in risk of hospitalization or death for the subject of about 1 to about 10 percent, such as from about 5 to about 7.5 percent, or about 6.8 percent. In further embodiments, administration of oral dosage forms may result in a relative reduction in risk of hospitalization or death for the subject of up to about 50 percent.

The present disclosure also relates to oral dosage forms for use as a medicament for prevention of viral infection or for providing antiviral prophylaxis, wherein the viral infection to be prevented is caused by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

The disclosure also provides oral dosage forms as described herein for use as a medicament for the prevention of viral infection or for providing antiviral prophylaxis, where the patient has previously (e.g., within 24 hours) been treated with another agent, which may be as described above.

In embodiments of the oral dosage forms as described herein for use as a medicament for the prevention of viral infection or for providing antiviral prophylaxis, the oral dosage forms are administered once daily, as a single dose. In particular aspects of these embodiments, the single daily dose may be provided as 1 to 400 2 mg individual pellets.

In embodiments of the oral dosage forms as described herein for use as a medicament for the prevention of viral infection or for providing antiviral prophylaxis, the oral dosage forms are administered twice daily, as two individual doses. In particular aspects of these embodiments, each individual dose may be provided as 1 to 400 2 mg individual pellets.

The disclosure further relates to oral dosage forms for use as a medicament for the prevention of viral infection or for providing antiviral prophylaxis, wherein the oral dosage forms are administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days.

The disclosure further relates to oral dosage forms for use as a medicament for the prevention of viral infection or for providing antiviral prophylaxis, wherein the oral dosage forms are administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days.

The disclosure further relates to oral dosage forms for use as a medicament for the prevention of viral infection or for providing antiviral prophylaxis, wherein the therapy commences prior to exposure to a viral infection or after a potential exposure to a viral infection. In specific embodiments, the oral dosage forms are administered beginning prior to potential exposure to viral infection. In other specific embodiments, the oral dosage forms are administered from 1 to 10 days after potential exposure to a viral infection, such as 1 day, 2 days, 3 days, 4 days, 5 days, 6 days, 7 days, 8 days, 9 days, or 10 days after potential exposure. In specific embodiments, the oral dosage forms are administered beginning less than 5 days after potential exposure, such as less than 1 day, 2 days, 3 days, 4 days, or 5 days after potential exposure.

The disclosure further relates to oral dosage forms for use as a medicament for the prevention of viral infection or for providing antiviral prophylaxis, wherein the oral dosage forms are administered to a subject who may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

Additionally, present disclosure relates to oral dosage forms for use as a medicament for the prevention of viral infection or for providing antiviral prophylaxis, wherein the oral dosage forms are administered to a subject who may have been exposed to a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of oral dosage forms as described herein for use as a medicament for the treatment of viral infection, where the patient has previously (e.g., within 24 hours) been treated with another agent, the oral dosage forms are administered once daily, as a single dose. In particular aspects of these embodiments, the single daily dose may be provided as 1 to 400 2 mg pellets.

In embodiments of pellets for use as a medicament for the treatment of viral infection, where the patient has previously (e.g., within 24 hours) been treated with another agent, the pellets are administered twice daily, as two individual doses. In particular aspects of these embodiments, each individual dose may be provided as 1 to 400 2 mg individual pellets.

### Combination Therapy

In another embodiment, the oral dosage forms as described herein can be combined with one or more additional therapeutic agents present methods useful for treating or preventing viral infection.

In one embodiment, the additional therapeutic agent is an antiviral agent.

In another embodiment, the additional therapeutic agent is an immunomodulatory agent, such as an immunosuppressive agent.

Accordingly, in one embodiment, the present disclosure provides methods for treating a viral infection in a subject, the method comprising administering to the subject: (i) the oral dosage forms as described herein, and (ii) at least one additional therapeutic agent, wherein the amounts administered are together effective to treat or prevent a viral infection.

When administering a combination therapy as described herein to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, oral dosage forms as described herein are administered during a time when the additional therapeutic agent(s) exert their prophylactic or therapeutic effect, or vice versa.

In another embodiment, oral dosage forms as described herein are administered in doses commonly employed when such agents are used as monotherapy for treating a viral infection.

In another embodiment, oral dosage forms as described herein and the additional therapeutic agent(s) are administered in doses lower than the doses commonly employed when such agents are used as monotherapy for treating a viral infection.

In still another embodiment, oral dosage forms as described herein and the additional therapeutic agent(s) act synergistically and are administered in doses lower than the doses commonly employed when such agents are used as monotherapy for treating a viral infection.

Viral infections and virus-related disorders that may be treated or prevented using the combination therapy methods as described therein include, but are not limited to, those listed above.

The oral dosage forms as described herein and additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of oral dosage forms as described herein and the additional therapeutic agent(s) may inhibit the resistance of a viral infection to these agents.

The therapies disclosed herein may be used in combination with one or more other active agents, including but not limited to, antiviral agents that are used in the prevention, treatment, control, amelioration, or reduction of risk of a particular disease or condition (e.g., viral infection). In one embodiment, a compound disclosed herein is combined with one or more other antiviral agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

When the therapies disclosed herein are used contemporaneously with one or more other active agents, oral dosage forms as described herein may be administered either simultaneously with, or before or after, one or more other active agent(s). Oral dosage forms as described herein may be administered separately, by the same or different route of administration, or together in the same pharmaceutical composition as the other agent(s).

The dosage amount of the oral dosage forms as described herein and of the one or more other active agent(s) may be varied and will depend upon the therapeutically effective dose. Generally, a therapeutically effective dose of each will be used. Combinations including oral dosage forms as described herein, and other active agents will generally include a therapeutically effective dose of each active agent. In such combinations, oral dosage forms as described herein and other active agents may be administered separately or in conjunction. In addition, the administration of one element may be prior to, concurrent with, or subsequent to the administration of other agent(s).

In one embodiment, this disclosure provides oral dosage forms as described herein, and at least one other active agent as a combined preparation for simultaneous, separate, or sequential use in therapy. In one embodiment, the therapy is the treatment of a viral infection, such as infection by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In aspects of this embodiment, the virus is selected from the group consisting of RSV, influenza A virus, influenza B virus, filoviridae, human coronavirus, SARS-CoV-1, MERS-CoV, SARS-CoV-2, and Ebola virus. In more specific aspects, the virus is selected from the group consisting of influenza A virus and influenza B virus. In other specific aspects, the virus is selected from the group consisting of human coronavirus, SARS-CoV-1, MERS-CoV, and SARS-CoV-2. In even more specific aspects, the virus is SARS-CoV-2.

In another embodiment, this disclosure provides oral dosage forms as described herein, and at least one other active agent as a combined preparation for simultaneous, separate, or sequential use in therapy. In one embodiment, the therapy is antiviral prophylaxis, such as for potential infection, either pre-exposure or post-exposure, by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In aspects of this embodiment, the virus is selected from the group consisting of RSV, influenza A virus, influenza B virus, filoviridae, human coronavirus, SARS-CoV-1, MERS-CoV, SARS-CoV-2, and Ebola virus. In more specific aspects, the virus is selected from the group consisting of influenza A virus and influenza B virus. In other specific aspects, the virus is selected from the group consisting of human coronavirus, SARS-CoV-1, MERS-CoV, and SARS-CoV-2. In even more specific aspects, the virus is SARS-CoV-2.

The disclosure also provides the use of oral dosage forms as described herein for treating a viral infection, where the patient has previously (e.g., within 24 hours) been treated with another agent.

The additional active agent(s) may be one or more agents selected from the group consisting of antiviral compounds, antigens, adjuvants, anti-cancer agents, CTLA-4 agonists, LAG-3 agonists, PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, antibiotics, anti-metabolites, retinoids, steroids, and immunomodulatory agents, including but not limited to antiviral vaccines. It will be understood the descriptions of the above additional active agents, and of those listed below, may be overlapping. It will also be understood that the treatment combinations are subject to optimization, and it is understood that the best combination to use of the antiviral nucleoside, and one or more additional active agents will be determined based on the individual patient needs.

Antiviral compounds that may be used in combination with the therapies disclosed herein include direct acting antivirals and antiviral compounds that target intracellular environments. In particular, antiviral compounds that may be used in combination with the therapies disclosed herein include antivirals that target SARS-CoV-2 virus (and COVID-19 caused by SARS-CoV-2 infection), influenza, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors. Such antiviral compounds include but are not limited to 2-DG, 2x-121, AB001, avifavir, AVM-0703, C21, CAL-02, CYTO-201 (naltrexone hydrochloride), Conronavir (TL-FVP-t), DW-2008S, DWJ-1248, elsufavirine, emtricitabine, eFT226, HP-163, IML-206, IMU-838, LAU-7b, MAN-19, MMS-019, OBP-2001, omega 3 viruxide, OPN-019, OYA-1, PP-001, PRTX-007, RBI-5000, RBT-9, RECCE529, RS-5614, SK11, SLV-213, T-COVID, TL-895, TYME-19, UCI-1, XC-221, fenretinide, nafamostat, nafamostat mesylate, nanomedivir (atazanavir/dexamethasone), nanofenretinide (ST-001), necuparanib (M-402), nelfinavir, nitazoxanide, piclidenoson, pixatimod, polyinosinic-plycytidylic acid, proxalutamide, hydrochloroquine, hydroxychloroquine, chloroquine, oseltamivir, oseltamivir phosphate, zanamivir, peramivir, baloxavir marboxil, remdesivir, favilavir, avifavir, favilavir/avifavir, vaniprevir, grazoprevir, elbasvir, narlaprevir, nitazoxanide, atazanavir, ritonavir, daclatasvir, farunavir, darunavir/cobicistat, saquinavir, indinavir, carfilzomib, ivaltinostat (CG200745), isotretinoin/tamoxifen, isotretinoin, tamoxifen, levamisole, prexasertib, ebselen, merimepodib, 1-deoxy-D-glucose prodrugs, formoterol, budesonide, rigosertib, erlotinib, silmitasertib, favipiravir, galidesivir, ledipasvir, lopinavir, lopinavir/ritonavir, levovir, tenofovir, and sofosbuvir, and combinations thereof.

Additional therapies that may be used in combination with the therapies disclosed herein include but are not limited to immunomodulators, such as interleukin 6 (IL-6) inhibitors, corticosteroids, TNF-inhibitors, and other immune-dependent therapies; antibody therapies, such as convalescent plasma therapies, hyperimmune globulin therapies, monoclonal antibodies, polyclonal antibodies, and neutralizing antibodies; soluble guanylate cyclase stimulator, such as riociguat; cannibidiols; and vaccines. The additional therapies contemplated include biological products that are biosimilar to any biological product or therapy expressly listed herein.

In particular, the additional therapies that may be used in combination with the therapies disclosed herein include but are not limited to 2,3,4,5,6-pentafluoro-N-(3-fluoro-4-methoxyphenyl)benzene sulfonamide, 3′,4′-didehydro-4′deoxy-8′-norvin-caleukoblastine, 47D11, 5-fluorouracil, abatacept, abiraterone acetate, ABX464, abibertinib, acalabrutinib, ACE2-Fc, ACE-MAB (STI-4920, CMAB020), acetylsalicylic acid, acetaminophen, ACT-20, Actemra, Actemra/RoActemra, adalimumab, adipose mesenchymal cells, AdMSCs (autologous adipose-derived stem cells), ADR-001, adrecizumab (HAM8101), ADX-629/reproxalap, AK-119, Alferon N, Allocetra (leukocyte cell based therapy), AlloStim, Allorx stem cells, ALT-100 (enamptcumab), ALT-803, Amnioboost, Ampion, altretamine, amiodarone, Anaferon, Anakinra, AMG-3777, anhydrovinblastine, anti-nCoV nanoviricides, aprepitant, AP-003 (AntiCovir), APL-9 (pegylated synthetic cyclic peptide), APX-115, AQCH, AR-701, ARO-COV, AS-1411, ascorbic acid, asunercept, atovaquone/azithromycin, AT-100 (rhSP-D), AT-301, AT-H201, ATI-450, ATR-002, auristatin, avdoralimab (IPH5401), axatilimab, AZD-1061, AZD-7442, alvelestat (AZD-9668), AZD-8895, azvudine, azvudine/tetrandrine, azithromycin, bardoxolone, bardoxolone methyl, baricitinib, BBT-032, bemcentinib, BGE-175, BIO-300, BIOMEDIVR, bevacizumab, bexarotene, bicalutamide, BIO-1106, BLD-2660, BLD-2736, BOLD-100, brequinar sodium, brilacidin, bromhexine hydrochloride, BTL-TML001, bleomycin, BMS-986253, BMS 184476, BT-086, BT-588, BXCL501, BXT-25, bucillamine, budesonide, cachectin, acalabrutinib, camrelizumab, camrelizumab/thymosin, captopril, CardioIRx, carrimycin, cavaltinib, comostat, camostat mesylate, canakinumab, CAP-1002, carboplatin, carmustine, CB5064 analogs, CD24Fc (recombinant fusion protein), cepharanthine, cemadotin, cenicriviroc, canthaquine, CERC-002, chlorambucil, chloropromazine, cholecalciferol, ciclesonide, cisplatin, ci-trimoxazole, CK-0802, clazakizumab, clarithromycin, CLBS-119, CM4620-IE, colchicine, CorLiCyte (umbilical cord lining stem cells), COVID-19 aptamer therapy, COVID-19 human mAb, COVID-19 neutralizing antibodies, COVID-19 siRNA therapy, COVID-HIG, COVID-EIG, spike glycoproteins, CoviGlobulin, COVI-GUARD (STI-1499), CPI-006, crizanlizumab, cryptophycin, CSL-324, CT-P59, CTAP-101, CV-15, CVL-218, cyclosporine, cell replacement therapies, cyclophosphamide, CYNK-001, cytarabine, dacarbazine, dactolisib, dactinomycin, dalargin, DAS-181, dapagliflozin, dapansurtrile, daunorubicin, decitabine, dexamethasone, DNL758 (SAR443122, RIPK 1 inhibitor), dipyridamole, DMX-200, DS-2319, deupirfenidone, duvelisib, DV-890, DWRX-2003, docetaxol, dolastatin, doxetaxel, doxorubicin (adriamycin), DP-710, EB-05, EB-201, ebastine, eculizumab, EDP-1815, efineptakin alfa, emapalumab, emtricitabine, ensifentrine, ENU-200, enoxaparin, enzalutamide, epaspire, etanercept, etoposide, eravacycline, famotidine, finasteride, fingolimod, flebogamma (IGIV31), fluvoxamine, foalumab (NI-0401, TZLS-401), fostamatinib, flutamide, FSD-201, FW 1022, FT516, Gamunex (IGIV-C), ganetespib, GC-376, Giapreza, GLS-1200, garadacimab, GC-5131A (hyperimmune globulin), GIGA-2050 rCIG), gimsilumab, GNS561, GP1681, GSK-2586881/APN-1, GSK-4182136, GTB-3550 (Trike 161533), haNK: CD-16, HB-adMSCs, HFB30132A, HLCM-051, heparin, hydrocortisone, hydroxyurea, ibuprofen, ibudilast (MN-166), icosapent ethyl, IC14, IDB-003, IFX-1/BDB-1, IgY-110, IMM101 IMS001, IMS002, ifosfamide, imatinib, infliximab, INM-005, interferon alfa, interferon alfa 1B, interferon alfa 2B, interferon beta 1A, interferon beta 1B, interleukin-6, interleukin-7, isoquercetin, itanapraced (CHF-5074), itolizumab, ivermectin, IVIG, JS012 (monoclonal antibody, LY-CoV016), jaktinib, kagocel, KB109, sarilumab, K-NK-ID101, KTH-222, lactoferrin, LAM-002A (apilimod dimesylate), lanadelumab, lamellasome, LB-1148, larazotide, leflunomide, lenzilumab, leronlimab (monoclonal antibody), levilimab (BCD-089), levamisole, liarozole, linagliptin, lipocurc, losartan, livilimab, lomustine (CCNU), lonidamine, losmapimod, lostartan, LY-CoV555 (LY-3819253), LY-3127804, mannitol, maraviroc, mastinib, mavrilimumab, MDV3100, mechlorethamine, MEDI-3506, melatonin, melphalan, meplazumab, merimepodib, Mesenchymal stem cells (MSCs), mesencure (cell replacement), metablok (anti-inflammatory), metformin, methotrexate, methylprednisolone, mitomycin, mivobulin isethionate, mosedipimod (EC-18), MP-0420, MP-0423, MRx4DP0004, N-acetylcysteine, N,N-dimethyl-L-valyl-L-valyl-N-methyl-L-valyl-L-prolyl-1-Lproline-t-butylamide, namilumab (IZN-101), nangibotide, narsoplimab, nebulized dornase alfa, NED-260, Niagen (nicotinamide riboside; Vitamin B3), NK cell therapy, niclosamide, nilutamide, nintedanib, nitric oxide, nivolumab, NL-CVX1, NLP-21, NP-02, N-120 (ifenprodil), novaferon, NT-17 (efineptakin alfa), NTR-441, octagam, olokizumab, omeprazole, onapristone, opaganib, OP-101, OT-101 (trabedersen), otilimab, ozanimod, paclitaxel, pacritinib, panaphix, pamrevlumab, paracetamol, PB1046, PTC299, pegylated interferon alpha, pegylated interferon alpha 2b, pegylated interferon lambda, pembrolizumab, PL-8177, pirfenidone, plitidepsin (aplidin), PneumoBlast, polyoxidonium, prazosin, prednimustine, prednisolone, prednisone, pritumumab, procarbazine, prolastin, PTC-299, pyronaridine/artesunate, radotinib, RAPA-501, ravulizumab, razuprotafib, interferon beta 1 agonists, RECC327, REGN-COV2 (antibody cocktail), reparixin, rintatolimod (ampligen), RLF-100 (aviptadil), RLS-0071, STI-5656 (abivertinib), Rhu-pGSN (gelsolin), rhizoxin, RPR109881, RoActemra, RUCONEST (conestat alfa), ruxolitinib, SAB-185, SAR443122, SARS-CoV-2 antibodies, SARS-CoV-2 monoclonal antibodies, SARS-CoV-2 polyclonal antibodies, SARS-CoV-2 neutralizing antibodies, SCTA01, Leukine (sargramostim), selenexor, sevoflurane, sertenef, siltuximab, sildenafil citate, silymarin, simvastatin, sirolimus, sirukumab, SIWA-318, solnatide, SNG-001, ST-266, stem cell educator therapy, STI-1499, STI-2020dna (COVI-MAB), STI-4398 (Covidtrap), stramustine phosphate, streptozocin, T cell therapies (TargNaturTa), TAK-671, TAK-888, TATX-36, TATX-99, TCB-007, TJ003234/TJM-2, TP508, TRV027, TD-0903, TLC19, tekrurna, tafenoquine, tamoxifen, tasonermin, taxanes, taxol, tetradrine, thalidomide, thimerosal, thymalfasin, tinzaparin, tocilizumab, tofacitinib, toremifene, tradipitant, tranexamic acid, trans sodium crocetinate (TSC), tramadol, tretinoin, TXA127 (antiotensin-(1-7) peptide), TY027, TZLS-501, UNI-911, ulinastatin, upamostat, vafidemstat, valsartan, icosapent ethyl, vazegepant, VBI-S, VERU-111, VHH72-Fc, vinblastine, vincristine, vindesine sulfate, vinflunine, VIR-2703 ALN-COV), VIR-7831, VIR-7832, Vitamin C, Vitamin D, XAV-19, Xpro-1595, XRx-101, zanubrutinib, zilucoplan, and zinc, and combinations thereof.

The additional vaccine therapies that may be used in combination with the therapies disclosed herein include but are not limited to inactivated vaccines, live-attenuated vaccines, recombinant vaccines, replication-deficient viral vector vaccines, mRNA-based vaccines, DNA vaccines, nanoparticle vaccines, non-replicating viral vectors, self-replicating RNA vaccines, self-amplifying RNA vaccines, protein subunit vaccines, Ii-Key peptide COVID-19 vaccines, gp96-based vaccines, intranasal vaccines, and mRNA lipid nanoparticle (mRNA-LNP) vaccine. In particular, the additional vaccine therapies that may be used in combination with the therapies disclosed herein include but are not limited to 7HP-349, AAVCOVID (gene-based vaccine), Ad26.COV2-S(non-replicating viral vector), Ad5-nCoV (recombinant vaccine; adenovirus type 5 vector), Ad5-S-nb2, AdCOVID (intranasal vaccine), AdimrSC-2F (protein subunit vaccine), AG0301-COVID19 (DNA vaccine), AKS-446, ARCOV, AV-COVID-19, AVI-205, AZD1222 (replication-deficient viral vector vaccine (adenovirus from chimpanzees)), Bacillus Calmette-Guerin (BCG) vaccine (live attenuated vaccines), bacTRL-Spike (monovalent oral vaccine (bifidobacterial)), BBIBP-CorV (inactivated vaccine), BC-PIC COVID-19 vaccine, BNT-162 (mRNA-based vaccine), BVX-0320, CDX-005, ChAd-SARS-CoV-2-S(adenovirus-based vaccine, Chimigen vaccine, CIGB-2020, CiVax, Coravax, CoroFlu, COV001/AZD-1222), Corona Vac (inactivated vaccine (formalin with alum adjuvant)), Corvax, CORVax-12, COVAX-19 (monovalent recombinant protein vaccine), Covaxin (BBV-152, inactivated vaccine), CoVepiT, CVnCoV (mRNA-based vaccine), DPX-COVID-19, DS-5670, E-6020, ELI-005, Epi VacCorona, EPV-CoV19, Flowvax, GC-004/Covax-19, GRAd-COV2 (adenovirus-based vaccine), GX-19 (DNA vaccine), HaloVax (self-assembling vaccine), HDT-301 (RNA vaccine), iBIO-201, IK-15800, INO-4700, INO-4800 (DNA vaccine (plasmid)), IPT-001, ISR-50, KBP-COVID-19, LEAPS COVID-19 vaccine, LineaDNA (DNA vaccine), LNP-mRNA, LNP-nCoVsaRNA, LUNAR-COVV19 (ARCT-021; self-replicating RNA vaccine), mRNA-1273 (mRNA-based vaccine), MAPS vaccine, MT-2766, MV-014-210, MVA-S, MVC-COV-1901, NVX-CoV233 (nanoparticle vaccine), NVX-CoV2373, Oncoquest, OraCOV, PDS-0203, PDS-0204, PiCoVacc, PittCoVacc (Recombinant protein subunit vaccine (delivered through microneedle array)), PolyPEPI-SCOV-2, QAZCOVID-IN, repRNA-CoV2S (LION/repRNA-CoV2S), RNAi vaccine, RV-1730m, rVSV COVID-19 vaccine, S-268019, Saponin vaccine adjuvant, SCB-2019 (protein subunit vaccine), Shingrix vaccine (GSK-1437173A), Sputnik-V (Gam-COVID-Vac), STI-6991, T-COVID™ (intranasal vaccine), TaliCoVax-19, TerraCoV2, Tertomotide (GV-1001), Tiba-pitt RNA vaccine, TNX-1800, TNX-1810, TNX-1820, TNX-2300, T-VIVA-19, V-590 (recombinant vaccine (vesicular stomatitis virus)), V-591 (measles vector vaccine), VBI-2900, VBI-2901, VBI-2902, VLA2001, Vivagel (SPL-7013), YF17D vector, ZIP-1642, and ZyCOV-D (DNA vaccine (plasmid)), and combinations thereof.

### Uses

The present disclosure also relates to uses of the oral dosage forms described herein to treat a viral infection in a subject in need thereof, wherein the viral infection is an infection by a virus selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B, filoviridae, human coronavirus, SARS-CoV-1, MERS-CoV, SARS-CoV-2, Ebola virus, and Zika virus. In a second aspect of these embodiments, the virus is selected from the group consisting of RSV, influenza A virus, influenza B virus, filoviridae, human coronavirus, SARS-CoV-1, MERS-CoV, SARS-CoV-2, and Ebola virus. In specific aspects, the virus is selected from the group consisting of influenza A virus and influenza B virus. In further specific aspects, the virus is selected from the group consisting of human coronavirus, SARS-CoV-1, MERS-CoV, and SARS-CoV-2. In specific embodiments, the virus is SARS-CoV-2.

In embodiments of the uses disclosed herein, the oral dosage forms are administered once daily, as a single dose. In particular aspects of these embodiments, the single daily dose may be provided as 1 to 400 2 mg individual pellets.

In embodiments of the uses disclosed herein, the oral dosage forms are administered twice daily, as two individual doses. In particular aspects of these embodiments, each individual dose may be provided as 1 to 400 2 mg individual pellets.

In embodiments of the uses disclosed herein, the oral dosage forms are administered once daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered once daily for 3 to 6 days, such as for 3 days, 4 days, 5 days, or 6 days. In another specific embodiment, the oral dosage forms are administered once daily for 5 days.

In embodiments of the uses disclosed herein, the oral dosage forms are administered twice daily for 1 to 20 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days. In specific embodiments, the oral dosage forms are administered twice daily for 3 to 6 days, such as for 3 days, 4 days, 5 days, or 6 days. In another specific embodiment, the oral dosage forms are administered twice daily for 5 days.

The disclosure further relates to uses of the oral dosage forms described herein to treat a viral infection in a subject in need thereof, wherein the use commences from 1 to 10 days after onset of symptoms of viral infection, such as 1 day, 2 days, 3 days, 4 days, 5 days, 6 days, 7 days, 8 days, 9 days, or 10 days after the onset of symptoms of viral infection. In specific embodiments, the oral dosage forms are administered beginning less than 5 days after onset of symptoms, such as less than 1 day, 2 days, 3 days, 4 days, or 5 days after onset of symptoms of viral infection. Symptoms of viral infection may include one or more of cough, sore throat, nasal congestion, runny nose, shortness of breath or difficulty breathing, muscle or body aches, fatigue/tiredness, feeling hot/feverish, chills, headache, nausea, vomiting, and diarrhea, although symptoms may vary by the type and severity of the viral infection. For example, symptoms of COVID-19 caused by SARS-CoV-2 viral infection may include one or more of cough, sore throat, nasal congestion, runny nose, shortness of breath or difficulty breathing, muscle or body aches, fatigue/tiredness, feeling hot/feverish, chills, headache, nausea, vomiting, diarrhea, loss of taste, and loss of smell.

The disclosure further relates to the above-described uses, wherein said subjects may be considered or is determined to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure further relates to the above-described uses, wherein said use reduces the risk of hospitalization or death for the subject. In embodiments, the use may result in a reduction in risk of hospitalization or death for the subject. In specific embodiments, the use may result in a reduction in risk of hospitalization or death for the subject of about 1 to about 10 percent, such as from about 5 to about 7.5 percent, or about 6.8 percent. In further embodiments, the use may result in a relative reduction in risk of hospitalization or death for the subject of up to about 50 percent.

Additionally, present disclosure relates to uses of the oral dosage forms described herein to prevent a viral infection in a subject in need thereof or to provide antiviral prophylaxis to a subject in need thereof (hereinafter “prophylactic use(s)”, wherein the viral infection is caused by virus is selected from the group consisting of Eastern equine encephalitis virus, Western equine encephalitis virus, Venezuelan equine encephalitis virus, Chikungunya virus, Ross River virus, orthomyxoviridae, paramyxoviridae, RSV, influenza A virus, influenza B virus, filoviridae, human coronavirus, SARS-CoV-1, MERS-CoV, SARS-CoV-2, Ebola virus, and Zika virus. In specific embodiments, the virus is selected from the group consisting of RSV, influenza A virus, influenza B virus, filoviridae, human coronavirus, SARS-CoV-1, MERS-CoV, SARS-CoV-2, and Ebola virus. In specific embodiments, the virus is selected from the group consisting of influenza A virus and influenza B virus. In specific embodiments, the virus is selected from the group consisting of human coronavirus, SARS-CoV-1, MERS-CoV, and SARS-CoV-2. In specific embodiments, the virus is SARS-CoV-2.

In embodiments of the prophylactic uses disclosed herein, the oral dosage forms are administered once daily, as a single dose. In particular aspects of these embodiments, the single daily dose may be provided as 1 to 400 2 mg individual pellets.

In embodiments of the prophylactic uses disclosed herein, the oral dosage forms are administered twice daily, as two individual doses. In particular aspects of these embodiments, each individual dose may be provided as 1 to 400 2 mg individual pellets.

The disclosure further relates to prophylactic uses, wherein the oral dosage forms are administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered once daily for 3 to 14 days, such as for 3 days, 4 days, 5 days, 6 days, 7 days, 8 days, 9 days, 10 days, 11 days, 12 days, 13 days, or 14 days.

The disclosure further relates to prophylactic uses, wherein the oral dosage forms are administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the oral dosage forms are administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the oral dosage forms are administered twice daily for 3 to 14 days, such as for 3 days, 4 days, 5 days, 6 days, 7 days, 8 days, 9 days, 10 days, 11 days, 12 days, 13 days, or 14 days.

The disclosure further relates to prophylactic uses, wherein the use commences prior to exposure to a viral infection or after a potential exposure to a viral infection. In specific embodiments, the oral dosage forms are administered beginning prior to potential exposure to viral infection. In other specific embodiments, the oral dosage forms are administered from 1 to 10 days after potential exposure to a viral infection, such as 1 day, 2 days, 3 days, 4 days, 5 days, 6 days, 7 days, 8 days, 9 days, or 10 days after potential exposure. In specific embodiments, the oral dosage forms are administered beginning less than 5 days after potential exposure, such as less than 1 day, 2 days, 3 days, 4 days, or 5 days after potential exposure.

The disclosure further relates to the above-described prophylactic uses, wherein said subjects may be considered to be at increased risk for severe illness from COVID-19. Such individuals may have one or more underlying medical condition associated with being at increased risk for severe illness from COVID-19, such as age greater than 60 years; active cancer (excluding minor cancers not associated with immunosuppression or significant morbidity/mortality (e.g., basal cell carcinomas)); chronic kidney disease (excluding participants on dialysis or has reduced eGFR <30 mL/min/1.73 m²); chronic obstructive pulmonary disease; obesity (body mass index of 30 or higher, where body mass index=weight (kg)/(height (m))²); serious heart conditions (heart failure, coronary artery disease, or cardiomyopathies); and/or diabetes mellitus. In one embodiment, the subject has not been vaccinated against COVID-19. In another embodiment, the subject has been vaccinated against COVID-19.

The disclosure relates to prophylactic uses as described above, wherein said method reduces the risk of hospitalization or death for the subject. In embodiments, the method may result in a reduction in risk of hospitalization or death for the subject. In specific embodiments, the method may result in a reduction in risk of hospitalization or death for the subject of about 1 to about 10 percent, such as from about 5 to about 7.5 percent, or about 6.8 percent. In further embodiments, the method may result in a relative reduction in risk of hospitalization or death for the subject of up to about 50 percent.

Many patients with COVID-19 recover with no or minimal medical intervention. However, clinical progression to severe disease severely impacts both patients and healthcare systems, increasing an individual's risks of requiring mechanical ventilation and of death and potentially overburdening hospital capacity and available healthcare resources during COVID-19 surges. Reducing the number of patients requiring hospitalization for COVID-19 is therefore critical. Vaccination remains by far the most important medical intervention available to reduce the risk of hospitalization or death from COVID-19. However, early treatment soon after symptom onset has also been shown as effective. The monoclonal antibodies bamlanivimab/etesevimab, casirivimab/imdevimab, and sotrovimab are currently the only treatments authorized for at-risk outpatients with COVID-19. Because monoclonal antibodies require administration via infusion or injection in a medical setting, a direct-acting, oral agent such as the herein described oral dosage forms that can be administered at home after diagnosis to pediatric patients or patients requiring flexible dosing or who have difficulty swallowing conventional forms may be more practical for non-hospitalized patients and would be an important new tool in treating COVID-19 caused by SARS-CoV-2.

Additional embodiments of the disclosure include the pharmaceutical compositions, combinations, uses, and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### EXAMPLES

The following examples are meant to be illustrative and should not be construed as further limiting. The contents of the figures and all references, patents, and published patent applications cited throughout this application are expressly incorporated herein by reference.

### Reference Example 1: Compound A Capsule Formulation

The capsule formulation for Compound A uses a wet granulated mixture filled into size 0 hypromellose capsules at a dose strength of 200 mg (fill weight of 285.7 mg). The composition of the adult capsule formulation is shown in Table 1. The excipients used in the drug product are commonly used in pharmaceutical manufacturing.

TABLE 1

| Component | Unit Strength (mg/capsule) |
|---|---|
| Compound A | 200.0 |
| Microcrystalline Cellulose | 67.1 |
| Hydroxypropyl Cellulose | 8.6 |
| Croscarmellose Sodium | 8.6 |
| Magnesium Stearate | 1.4 |
| Total Fill Weight | 285.7 |
| Hypromellose capsule, Swedish Orange | 1 Capsule |
| Opaque, Size 0 | |

### Example 1: Compound A Pellets

Pellets were prepared by dispensing the following ingredients into a blender vessel preserving the proportions as indicated in Table 2. The ingredients were blended. Then, the blend was granulated by roller compaction. The granules were added to a blender vessel with magnesium stearate added, and the ingredients were blended. Subsequently, the blend was compressed into pellets.

TABLE 2

| | Formulation | | | | | | |
|---|---|---|---|---|---|---|---|
| Ingredients | A | B | C | D | E | F | G |
| Compound A | 2.00 | 2.00 | 2.00 | 2.00 | 2.00 | 2.00 | 2.00 |
| | mg | mg | mg | mg | mg | mg | mg |
| Microcrystalline | 3.98 | 3.95 | 3.92 | 3.85 | 3.96 | 3.95 | 3.85 |
| Cellulose | mg | mg | mg | mg | mg | mg | mg |
| Tri-Basic | — | 0.0300 | 0.0600 | 0.130 | — | — | — |
| Calcium | | mg | mg | mg | | | |
| Phosphate | | | | | | | |
| SiO₂ | — | — | — | — | 0.0163 | 0.0300 | 0.130 |
| | | | | | mg | mg | mg |
| Croscarmellose | 0.390 | 0.390 | 0.390 | 0.390 | 0.390 | 0.390 | 0.390 |
| Sodium | mg | mg | mg | mg | mg | mg | mg |
| Magnesium | 0.130 | 0.130 | 0.130 | 0.130 | 0.130 | 0.130 | 0.130 |
| Stearate | mg | mg | mg | mg | mg | mg | mg |
| Image (mg) | 6.50 | 6.50 | 6.50 | 6.50 | 6.50 | 6.50 | 6.50 |
| | mg | mg | mg | mg | mg | mg | mg |

As demonstrated in Table 3, inclusion of a glidant improved the flow properties of the blend; Table 3 shows the results of a quantitative assessment of flow data (“flodex”), evaluating wide range of SiO₂ (0.00%-2.00%) and tri-basic calcium phosphate (0.00%-2.00%). In general, materials having flodex values greater than 28 mm are considered poor flowing; materials having flodex values between 16 mm and 28 mm are considered moderately cohesive; and materials having flodex values less than 16 mm are considered good flowing. For roller compaction, a flodex value less than 30 is recommended. As can be seen from Table 3, inclusion of a glidant improves the flow of the blend so that it is suitable for roller compaction to form pellets. The use of tri-basic calcium phosphate as a glidant improves flow of the blend and process robustness, which ultimately has an impact on the critical quality attributes of granules.

TABLE 3

Formulation

| | A | B | C | D | E | F | G |
|---|---|---|---|---|---|---|---|
| Flodex (±2 mm) | 31 | 26 | 22 | 25 | 27 | 28 | 27 |

### Example 2: Compound A Pellets Having Varying Image Sizes

Pellets were prepared by dispensing the following ingredients into a blender vessel preserving the following proportions as indicated in Table 4. The ingredients were blended. Then, the blend was granulated by roller compaction. The granules were added to a blender vessel with magnesium stearate added, and the ingredients were blended. Subsequently, the blend was compressed on a tablet press into pellets.

TABLE 4

| | Formulation | | | | | | | | | |
|---|---|---|---|---|---|---|---|---|---|---|
| Ingredients | D | | H | | I | | J | | K | |
| Compound A | 2.00 | mg | 0.0650 | mg | 5.20 | mg | 0.308 | mg | 3.08 | mg |
| Microcrystalline | 3.85 | mg | 5.79 | mg | 0.650 | mg | 0.592 | mg | 5.92 | mg |
| Cellulose | | | | | | | | | | |
| Tri-Basic | 0.130 | mg | 0.130 | mg | 0.130 | mg | 0.0200 | mg | 0.200 | mg |
| Calcium | | | | | | | | | | |
| Phosphate | | | | | | | | | | |
| Croscarmellose | 0.390 | mg | 0.390 | mg | 0.390 | mg | 0.0600 | mg | 0.600 | mg |
| Sodium | | | | | | | | | | |
| Magnesium | 0.1300 | mg | 0.1300 | mg | 0.1300 | mg | 0.0200 | mg | 0.2000 | mg |
| Stearate | | | | | | | | | | |
| Image (mg) | 6.50 | mg | 6.50 | mg | 6.50 | mg | 1.00 | mg | 10.0 | mg |

As the levels of Compound A increases in the formulation, the flow properties of the pre-roller compaction powder blend worsens due to the cohesive nature of the material. In addition, although Compound A has good aqueous solubility, it exhibits poor wettability in aqueous media. As the levels of Compound A increases in the formulation, the property of the powder blend is dominated by the properties of Compound A, resulting in poor wettability, dispersibility and uniformity in water. Accordingly, the proportion of Compound A in the formulations having desirable properties is limited.

### Example 3: Compound A Pellets Containing Varying Amounts of Croscarmellose Sodium and Magnesium Stearate

Pellets were prepared by dispensing the following ingredients into a blender vessel preserving the following proportions as indicated in Table 5. The ingredients were blended. Then, the blend was granulated by roller compaction. The granules were added to a blender vessel with magnesium stearate added, and the ingredients were blended. Subsequently, the blend was compressed on a tablet press into pellets.

TABLE 5

| | Formulation | | | | | | | | | |
|---|---|---|---|---|---|---|---|---|---|---|
| Ingredients | D | | L | | M | | N | | O | |
| Compound A | 2.00 | mg | 2.00 | mg | 2.00 | mg | 2.00 | mg | 2.00 | mg |
| Microcrystalline | 3.85 | mg | 4.22 | mg | 3.66 | mg | 3.95 | mg | 3.66 | mg |
| Cellulose | | | | | | | | | | |
| Tri-Basic | 0.13 | mg | 0.13 | mg | 0.13 | mg | 0.13 | mg | 0.13 | mg |
| Calcium | | | | | | | | | | |
| Phosphate | | | | | | | | | | |
| Croscarmellose | 0.390 | mg | 0.020 | mg | 0.580 | mg | 0.390 | mg | 0.390 | mg |
| Sodium | | | | | | | | | | |
| Magnesium | 0.130 | mg | 0.130 | mg | 0.130 | mg | 0.030 | mg | 0.30 | mg |
| Stearate | | | | | | | | | | |
| Image (mg) | 6.50 | mg | 6.500 | mg | 6.500 | mg | 6.50 | mg | 6.50 | mg |

Croscarmellose sodium, a hydrophilic agent, is added to the formulation. The level of croscarmellose sodium does not have a notable impact on physical attributes of the powder blend. With varying amounts of croscarmellose sodium, the dissolution behavior of the pellets was not significantly impacted due to the high surface area, which facilitates rapid disintegration. For younger pediatric patients, such as infants, the pellets can be dissolved in water (or other suitable liquid) and administered. The level of disintegrant has an impact of the size and uniformity of the dispersed particles in aqueous media. This allows improved performance and ease of dosing either by having a patient swallow the aqueous dispersion or by administering the aqueous dispersion via nasogastric tube.

Magnesium stearate is used as a lubricant both intragranularly (after blending the pre-granulated blend) and extragranularly (after roller compaction). The intragranular lubrication is performed to minimize sticking of the powder blend to the roll surfaces during roller compaction. The resulting pellets are lubricated to prevent sticking of the pellets during compression process. Varying levels of magnesium stearate did not have a notable effect on in vitro dissolution performance. However, the hardness of the pellets decreases with increasing levels of magnesium stearate, resulting in less robust formulation.

### Example 4: Compound A Pellets Containing Hydroxypropyl Cellulose and Varying Amounts of Compound A

Pellets were prepared by dispensing the following ingredients (with the exception of magnesium stearate) into a wet granulation bowl preserving the following proportions as indicated in Table 6. Microcrystalline cellulose may be added intragranularly, extragranularly, or both intragranularly andextragranularly. The ingredients were mixed and subsequently granulated with water. The resulting wet granules were deagglomerated, dried and milled to achieve a suitable particle size. The granules were added to a blender vessel with magnesium stearate added, and blended. Subsequently, the blend was compressed on a tablet press into pellets.

TABLE 6

| | Formulation | | | | | | | | | |
|---|---|---|---|---|---|---|---|---|---|---|
| Ingredients | D | | P | | Q | | R | | S | |
| Compound A | 2.00 | mg | 2.00 | mg | 2.00 | mg | 2.00 | mg | 4.00 | mg |
| Microcrystalline | 3.85 | mg | 4.08 | mg | 3.98 | mg | 3.79 | mg | 1.98 | mg |
| Cellulose | | | | | | | | | | |
| Tri-Basic | — | | 0.0950 | mg | 0.190 | mg | 0.390 | mg | 0.195 | |
| Calcium | | | | | | | | | | |
| Phosphate | | | | | | | | | | |
| Croscarmellose | 0.130 | mg | — | | — | | — | | — | |
| Sodium | | | | | | | | | | |
| Magnesium | 0.390 | mg | 0.195 | mg | 0.195 | mg | 0.195 | mg | 0.195 | mg |
| Stearate | | | | | | | | | | |
| Image (mg) | 0.130 | mg | 0.130 | mg | 0.130 | mg | 0.130 | mg | 0.130 | mg |

Hydroxypropyl cellulose is commonly used as a binder that facilitates the formulation of granules during wet granulation. It effectively promotes controlled granule growth while also allowing for rapid dissolution of the drug product. Binder level can affect particle size distribution of the resultant granulation and dissolution rates of the compressed minitablets. Particle size of granules increases with increasing levels of hyroxypropyl cellulose. The dissolution behavior of the pellets was not significantly impacted by varying levels of binder, due to the high surface area of the pellets. However, with increasing level of binder, the rate at which disintegrated particles disperse to create a uniform suspension decreases.

As the levels of Compound A increases in the formulation, the property of the powder blend is dominated by the properties of Compound A, resulting in poor wettability, dispersibility and uniformity in water. Accordingly, the proportion of Compound A in the formulations having desirable properties is limited.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. An oral dosage form comprising an antiviral nucleoside, one or more compression aid, one or more glidant, one or more lubricant, and one or more disintegrant, wherein
(a) the oral dosage form is a pellet having a diameter of less than or equal to 4.0 mm, and
(b) the antiviral nucleoside is selected from prodrugs of β-D-N(4)-hydroxycytidine,

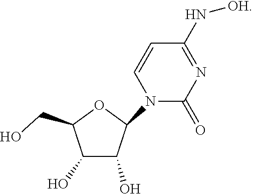 [SMILES: O=c1nc(NO)ccn1[C@@H]1O[C@H](CO)[C@@H](O)[C@H]1O]

2. The oral dosage form according to claim 1, wherein the antiviral nucleoside is {(2R,3S,4R,5R)-3,4-dihydroxy-5-[4-(hydroxyimino)-2-oxo-3,4-dihydropyrimidin-1 (2H)-yl] oxolan-2-yl}methyl 2-methylpropanoate): 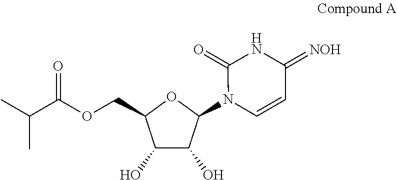
or a pharmaceutically acceptable salt, tautomer, or prodrug thereof.

3. The oral dosage form according to claim 2, wherein the antiviral nucleoside is Compound A.

4. The oral dosage form according to claim 2, wherein the antiviral nucleoside is a prodrug of Compound A.

5. The oral dosage form according to claim 3, wherein the pellet has a diameter in a range of from about 1.00 mm to about 4.00 mm.

6. The oral dosage form according to claim 5, wherein the pellet has a diameter of about 2.00 mm.

7. The oral dosage form according to claim 3, wherein the pellet has a weight in a range of from about 1.00 mg to about 10.0 mg.

8. The oral dosage form according to claim 7, wherein the pellet has a weight of about 6.50 mg.

9. The oral dosage form according to claim 3, wherein the pellet comprises the antiviral nucleoside in an amount of from about 0.0650 mg to about 5.20 mg.

10. The oral dosage form according to claim 9, wherein the pellet comprises the antiviral nucleoside in an amount of about 2.00 mg.

11. The oral dosage form according to claim 3, wherein the pellet comprises Compound A in an amount relative to the total weight of the pellet of from about 10.0 wt % to about 80.0 wt %.

12. The oral dosage form according to claim 11, wherein the pellet comprises the antiviral nucleoside in an amount relative to the total weight of the pellet of about 30.8 wt %.

13. The oral dosage form according to claim 3, wherein the one or more compression aid is selected from the group consisting of directly compressible starches, dibasic calcium phosphate, spray-dried lactose, anhydrous lactose, spray crystallized maltose, spray crystallized dextrose, crystalline sorbitol, mannitol, sucrose, microfine cellulose, and microcrystalline cellulose.

14. The oral dosage form according to claim 13, wherein the one or more compression aid is selected from directly compressible starches, dibasic calcium phosphate, spray-dried lactose, anhydrous lactose, mannitol, microfine cellulose, and microcrystalline cellulose.

15. The oral dosage form according to claim 13, wherein the one or more compression aid is microcrystalline cellulose.

16. The oral dosage form according to claim 13, wherein the one or more compression aid is present in an amount of from about 0.195 mg to about 6.00 mg.

17. The oral dosage form according to claim 16, wherein the one or more compression aid is present in an amount of about 3.92 mg.

18. The oral dosage form according to claim 13, wherein the one or more compression aid is present in an amount relative to the total weight of the pellet of from about 3.00 wt % to about 92.3 wt %.

19. The oral dosage form according to claim 18, wherein the one or more compression aid is present in an amount relative to the total weight of the pellet of about 60.2 wt %.

20. The oral dosage form according to claim 3, wherein the one or more glidant selected from starch, corn starch, talc, silica, colloidal silica, and tri-basic calcium phosphate.

21. The oral dosage form according to claim 20, wherein the one or more glidant is tri-basic calcium phosphate.

22. The oral dosage form according to claim 20, wherein the one or more glidant is present in an amount of from about 0.016 mg to about 0.195 mg.

23. The oral dosage form according to claim 22, wherein the one or more glidant is present in an amount of about 0.0650 mg.

24. The oral dosage form according to claim 20, wherein the one or more glidant is present in an amount relative to the total weight of the pellet of from about 0.250 wt % to about 3.00 wt %.

25. The oral dosage form according to claim 24, wherein the one or more glidant is present in an amount relative to the total weight of the pellet of about 1.00 wt %.

26. The oral dosage form according to claim 3, wherein the one or more lubricant is selected from magnesium silicate, calcium stearate, stearic acid, talc, sodium lauryl sulfate, magnesium lauryl sulfate, and magnesium stearate.

27. The oral dosage form according to claim 26, wherein the one or more lubricant is magnesium stearate.

28. The oral dosage form according to claim 27, wherein the one or more lubricant is extragranular magnesium stearate.

29. The oral dosage form according to claim 26, wherein the one or more lubricant is present in an amount of from about 0.0325 mg to about 0.3250 mg.

30. The oral dosage form according to claim 29, wherein the one or more lubricant is present in an amount of about 0.130 mg.

31. The oral dosage form according to claim 26, wherein the one or more lubricant is present in an amount relative to the total weight of the pellet of from about 0.500 wt % to about 5.00 wt %.

32. The oral dosage form according to claim 31, wherein the one or more lubricant is present in an amount relative to the total weight of the pellet of about 2.00 wt %.

33. The oral dosage form according to claim 3, wherein the one or more disintegrant is selected from calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, low-substituted hydroxypropyl cellulose, hydroxypropyl starch, magnesium aluminum silicate, methylcellulose, sodium alginate, starch, sodium starch glycolate, and croscarmellose sodium.

34. The oral dosage form according to claim 33, wherein the one or more disintegrant is selected from calcium alginate, calcium sodium alginate, calcium carboxymethylcellulose, calcium cellulose glycolate, carmellose calcium, crospovidone, microcrystalline cellulose, powdered cellulose, chitosan hydrochloride, corn starch, pregelatinized starch, low-substituted hydroxypropyl cellulose, hydroxypropyl starch, magnesium aluminum silicate, methylcellulose, sodium alginate, starch, sodium starch glycolate, and croscarmellose sodium.

35. The oral dosage form according to claim 33, wherein the one or more disintegrant is croscarmellose sodium.

36. The oral dosage form according to claim 33, wherein the one or more disintegrant is present in an amount of from about 0.0163 mg to about 0.585 mg.

37. The oral dosage form according to claim 36, wherein the one or more disintegrant is present in an amount of about 0.390 mg.

38. The oral dosage form according to claim 33, wherein the one or more disintegrant is present in an amount relative to the total weight of the pellet of from about 0.250 wt % to about 9.00 wt %.

39. The oral dosage form according to claim 38, wherein the one or more disintegrant is present in an amount relative to the total weight of the pellet of about 6.00 wt %.

40. The oral dosage form according to claim 39, further comprising one or more binders.

41. The oral dosage form according to claim 40, wherein the one or more binder is selected from starches, cellulose, ethylcellulose, cellulose acetate, carboxymethyl cellulose calcium, sodium carboxymethyl cellulose, methylcellulose, hydroxypropyl methylcellulose, hydroxypropyl cellulose, polyvinyl pyrrolidone, and mixtures thereof, and fillers, wherein the filler is selected from microcrystalline cellulose, powdered cellulose, mannitol, lactose, calcium phosphate, starch, pre-gelatinized starch, and mixtures thereof.

42. The oral dosage form according to claim 40, wherein the one or more binder is selected from starches, cellulose, ethylcellulose, cellulose acetate, carboxymethyl cellulose calcium, sodium carboxymethyl cellulose, methylcellulose, hydroxypropyl methylcellulose, hydroxypropyl cellulose, polyvinyl pyrrolidone, microcrystalline cellulose, powdered cellulose, mannitol, lactose, calcium phosphate, starch, pre-gelatinized starch, and mixtures thereof.

43. The oral dosage form according to claim 40, wherein the one or more binder is hydroxypropyl cellulose.

44. The oral dosage form according to claim 40, wherein the one or more binder is present in an amount of from about 0.000 mg to about 0.3900 mg.

45. The oral dosage form according to claim 44, wherein the one or more binder is present in an amount of about 0.1950 mg.

46. The oral dosage form according to claim 40, wherein the one or more binder is present in an amount relative to the total weight of the pellet of from about 0.000 wt % to about 6.000 wt %.

47. The oral dosage form according to claim 46, wherein the one or more binder is present in an amount relative to the total weight of the pellet of about 3.000 wt %.

48. An oral dose comprising from about 1 to about 400 pellets of the oral dosage form according to claim 3.

49. The oral dose according to claim 48, wherein the pellets are provided in a sachet.

50. The oral dose according to claim 48, wherein the oral dosage form is provided as pellets in a capsule.
